(19) United States
(12) Patent Application Publication
(10) Pub. No.: US 2013/0136696 A1
(43) Pub. Date: May 30, 2013

(54) METALLIC NANOPARTICLES WITH COATED SHELLS AND APPLICATIONS OF SAME

(71) Applicant: Board of Trustees of the University of Arkansas, Little Rock, AR (US)

(72) Inventors: Alexandru S. Biris, Little Rock, AR (US); Yang Xu, Little Rock, AR (US); Zhongrui Li, Little Rock, AR (US); Alexandru R. Biris, Cluj Napoca (RO)

(73) Assignee: BOARD OF TRUSTEES OF THE UNIVERSITY OF ARKANSAS, Little Rock, AR (US)

(21) Appl. No.: 13/745,282

(22) Filed: Jan. 18, 2013

Related U.S. Application Data
(62) Division of application No. 12/606,958, filed on Oct. 27, 2009.
(60) Provisional application No. 61/197,405, filed on Oct. 27, 2008.

(51) Int. Cl. A61K 9/14; A61K 51/02

(52) U.S. Cl. CPC A61K 9/141; A61K 51/02
USPC 424/9.3; 424/490; 514/7.6; 424/130.1; 514/44 R

(57) ABSTRACT

A process or method for treating cancer. In one embodiment, the method includes the steps of providing a plurality of metallic nanoparticles, wherein each of the plurality of metallic nanoparticles has a core formed with a first metallic material, and a shell formed with a non-metallic material containing carbon, and wherein the shell is formed to enclose the metallic core completely, introducing said metallic nanoparticles into a mammal such that said metallic nanoparticles selectively target at least one type of cancerous cell, and subsequently applying at least one radio frequency of electromagnetic waves to said mammal for a period of time effective to induce skin currents in the cores of the first metallic material of said metallic nanoparticles to cause heat generated locally around targeted at least one type of cancerous cell to kill said cancerous cell.

### CROSS-REFERENCE TO RELATED PATENT APPLICATION

This application is a divisional application of, and claims the benefit of, U.S. patent application Ser. No. 12/606,958, filed Oct. 27, 2009, entitled “METALLIC NANOPARTICLES WITH COATED SHELLS AND APPLICATIONS OF THE SAME,” by Alexandru S. Biris, et al., which itself claims the benefit of, pursuant to 35 U.S.C. §119(e), U.S. Provisional Patent Application Ser. No. 61/197,405, filed Oct. 27, 2008, entitled “METALLIC NANOPARTICLES COATED WITH GRAPHITIC SHELLS AS LOCALIZED RADIO FREQUENCY ABSORBERS FOR CANCER THERAPY,” by Alexandru S. Biris et al. The above identified applications are incorporated herein in its entirety by reference.

Some references, which may include patents, patent applications and various publications, are cited and discussed in the description of this invention. The citation and/or discussion of such references is provided merely to clarify the description of the present invention and is not an admission that any such reference is “prior art” to the invention described herein. All references cited and discussed in this specification are incorporated herein by reference in their entireties and to the same extent as if each reference was individually incorporated by reference. In terms of notation, hereinafter, [n] represents the nth reference cited in the reference list. For example, [29] represents the 29th reference cited in the reference list, namely, Biris A R, Biris A S, Dervishi E, Lupu D, Trigwell S, Rahman Z and Marginean P 2006 Catalyst excitation by radio frequency for improved carbon nanotubes synthesis Chem. Phys. Lett. 429 204-8.

### FIELD OF THE INVENTION

The present invention relates generally to metallic nanoparticles, and more particularly to metallic nanoparticles with coated shells, and applications of same such as localized radio frequency absorbers for cancer therapy.

### BACKGROUND OF THE INVENTION

The use of nanoparticles in biology and medicine currently is one of the most intensely researched areas in nanotechnology. Nanoparticles are utilized very actively in drug delivery cancer cell diagnostics and therapeutics. Magnetic nanoparticles, especially, are employed in many areas of medical studies, such as contrast agents for magnetic resonance imaging (MRI) of biological tissues and processes and colloidal mediators for magnetic hyperthermia of cancer. Many methods have been developed to synthesize and stabilize a wide variety of nanoparticles. Their stability is one of the most important factors for their use in complex biological and medical applications.

However, most of the nanoparticles tend to aggregate together in order to reduce their surface free energy. On the other hand, nanoparticles can be easily oxidized in air, and therefore lose partially or completely desired properties, such as their surface reactivity, structural and magnetic characteristics, and their oxidative states. Direct contact between metallic nanoparticles and human tissues may also cause undesired consequences for the human tissue.

Therefore, a heretofore unaddressed need exists in the art to address the aforementioned deficiencies and inadequacies.

### SUMMARY OF THE INVENTION

The present invention, in one aspect, relates to a process or method for treating cancer. In one embodiment, the method includes the steps of providing a plurality of metallic nanoparticles, wherein each of the plurality of metallic nanoparticles has a core formed with a first metallic material, and a coated shell formed with a non-metallic material containing carbon, and wherein the coated shell is formed to enclose the metallic core completely; introducing said metallic nanoparticles into a mammal such that said metallic nanoparticles selectively target at least one type of cancerous cell; and subsequently applying at least one radio frequency of electromagnetic waves to said mammal for a period of time effective to induce skin currents in the cores of the first metallic material of said metallic nanoparticles to cause heat generated locally around targeted at least one type of cancerous cell to kill said cancerous cell.

In one embodiment, said at least one radio frequency of electromagnetic waves is adjusted to be absorbed by cores of the first metallic material of said metallic nanoparticles.

In one embodiment, said at least one radio frequency of electromagnetic waves is smaller than a frequency threshold.

In one embodiment, the frequency of electromagnetic waves radiation is in the range of radio frequency, preferably smaller than a frequency threshold of 500 KHz.

In one embodiment, the period of time is greater than a time threshold.

In one embodiment, the period of time effective is in a range of 4 minute to 20 minutes, more preferably between 6 minutes and 30 minutes, greater than a time threshold of 4 minutes.

In one embodiment, the first metallic material is selected from the group consisting of Sb, Li, Rb, Ti, V, Mn, Fe, Ni, Cu, Zn, Zr, Mo, Ru, Rh, Pd, Ag, W, Ir, Pt, and a combination thereof.

In one embodiment, the first metallic material is Co.

In one embodiment, the non-metallic material containing carbon is selected from the group consisting of carbon black, fullerene, graphite and carbon.

The present invention, in another aspect, relates to a process or method for treating cancer. In one embodiment, the method includes the steps of providing a plurality of nanostructures, wherein each of the plurality of nanostructures has a core formed with a first metallic material, and a coated shell formed with a second material that is different from the first metallic material, and wherein the coated shell is formed to enclose the metallic core; introducing said nanostructures into a mammal such that said nanostructures selectively target at least one type of cancerous cell; and subsequently applying at least one radio frequency of electromagnetic waves to said mammal for a period of time effective to induce skin currents in the cores of the first metallic material of said nanostructures to cause heat generated locally around targeted at least one type of cancerous cell to kill said cancerous cell.

In one embodiment, said at least one radio frequency of electromagnetic waves is adjusted to be absorbed by cores of the first metallic material of said nanostructures.

In one embodiment, said at least one radio frequency of electromagnetic waves is smaller than a frequency threshold of 500 KHz.

In one embodiment, the period of time is greater than a time threshold of 4 minutes.

In one embodiment, the first metallic material is selected from the group consisting of Co, Sb, Li, Rb, Ti, V, Mn, Fe, Ni, Cu, Zn, Zr, Mo, Ru, Rh, Pd, Ag, W, Ir, Pt, and a combination thereof.

In one embodiment, the second material is selected from the group consisting of non-metal materials containing carbon, noble metallic materials, and polymeric materials.

The present invention, in yet another aspect, relates to a nanostructure. In one embodiment, the nanostructure has a core formed with a first metallic material, wherein the core has a diameter in the range of 5 to 10 nm, and a shell formed with a second material that is different from the first metallic material, wherein the shell is formed to enclose the metallic core and has a thickness of at least two layers of atoms of said second material.

In one embodiment, said core is adapted to absorb at least one radio frequency of electromagnetic waves when said core is subject to the radiation of said electromagnetic waves.

In one embodiment, the nanostructure is usable as a localized RF absorber for cancer therapy.

In one embodiment, the first metallic material is selected from the group consisting of Co, Sb, Li, Rb, Ti, V, Mn, Fe, Ni, Cu, Zn, Zr, Mo, Ru, Rh, Pd, Ag, W, Ir, Pt, and a combination thereof.

In one embodiment, the second material is selected from the group consisting of non-metal materials containing carbon, noble metallic materials such as gold, silver and the like, and polymeric materials.

In one embodiment, the nanostructure is usable as a MRI contrast agent.

In one embodiment, the nanostructure is usable as a delivery vehicle for drug and biological systems that include growth factors, antibodies, genes, DNA, RNA and a combination of them to a targeted area. When used as a delivery vehicle for drug, for example, drugs can be attached to the nanostructures for targeted delivery.

In one embodiment, the core of the nanostructure is adapted to absorb laser radiation or electromagnetic radiation when said core is subject to the laser radiation or electromagnetic radiation, where the nanostructure acts as a photothermal or photoacoustic agent.

In one embodiment, the nanostructure can be coated with one or more polymeric nanostructures for better integration with biological systems.

These and other aspects of the present invention will become apparent from the following description of the preferred embodiment taken in conjunction with the following drawings, although variations and modifications therein may be affected without departing from the spirit and scope of the novel concepts of the disclosure.

### BRIEF DESCRIPTION OF THE DRAWINGS

FIG. 1 shows a schematic diagram of HeLa cell with C—Co-NPs apoptosis process under RF excitation according to one embodiment of the present invention.

FIG. 2 shows (a) Schematic diagram of a Co-NPs covered by two layers of graphitic carbon. (b) Low and high magnification TEM images of such nanostructures obtained by CCVD method. (c) Raman spectrum data of the C—Co-NPs. (d) XPS spectrum of the Co2p peaks and the inset represents the XRD pattern of C—Co-NPs.

FIG. 3 shows RF excitation setup (350 kHz, 5 kW) used for the thermal ablation of HeLa cells according to one embodiment of the present invention.

FIG. 4 shows (a) Cytotoxicity effects of the C—Co-NPs and the single-wall carbon nanotubes on the HeLa cancer cells without any RF exposure. There were observed no significant effects on the HeLa cells viability due to the RF treatment when the cells were not incubated with any nanoparticles. (b) Effect of different concentrations of C—Co-NPs (1) 0 μg ml⁻¹, (2) 0.83 μg ml⁻¹, (3) 1.66 μg ml⁻¹, (4) 2.5 μg ml⁻¹ on the amount of HeLa cells that died as a function of the exposure time of 350 kHz RF heating. (c) C—Co-NPs concentration effect on the HeLa cells death rates when exposed for 20 min to 350 kHz RF treatment. (d) Comparative RF induced temperature variations for the dispersions of C—Co-NPs in the media solutions and powders as a function of nanoparticles amount. (e) Percentage of killed HeLa cells with different concentration of internalized C—Co-NPs and SWNTs after 20 min of 350 kHz RF treatment.

FIG. 5 shows Fluorescence microscopy images of HeLa cells. (a) Control HeLa cells. (b) After 24 h incubation time, the C—Co-NPs were found to aggregate around and further penetrate into the nucleus of HeLa cells. (c) High magnification image of a HeLa cell nucleus surrounded by C—Co-NPs. (d) C—Co-NPs are being uptaken by the HeLa cells and they were found to cross and agglomerate inside the nucleus and cytoplasm. (e) Low magnification optical microscopy image indicating the nucleus fragmentation of HeLa cells incubated with C—Co-NPs and after a 350 kHz of RF heating for 20 min. (f) High magnification image indicating the nucleus fragmentation of HeLa cells incubated with C—Co-NPs and after a 350 kHz of RF heating for 20 min. (g) Low magnification optical image indicating the extensive death of the Co—NPs containing cells after they were exposed to the RF radiation for 20 min. The cells were stained in order to distinguish between the living (green for acridine orange) and the dead cells (orange for ethidium bromide).

FIG. 6 shows DNA fragmentation studies. (1) Marker DNA, (2) DNA extracted from HeLa cells without any nanoparticles and no RF exposure, (3) DNA of the HeLa cells incubated with SWNT and exposed to RF excitation, and (4) DNA of the HeLa cells incubated with C—Co-NPs and exposed to RF excitation.

FIG. 7 shows the magnetization curves of the C—Co-NPs (A), C—Fe-NPs (B) and C—Fe/Co-NPs (C), respectively.

### DETAILED DESCRIPTION OF THE INVENTION

The present invention is more particularly described in the following examples that are intended as illustrative only since numerous modifications and variations therein will be apparent to those skilled in the art. Various embodiments of the invention are now described in detail. Referring to the drawings, like numbers indicate like parts throughout the views. As used in the description herein and throughout the claims that follow, the meaning of “a,” “an,” and “the” includes plural reference unless the context clearly dictates otherwise. Also, as used in the description herein and throughout the claims that follow, the meaning of “in” includes “in” and “on” unless the context clearly dictates otherwise. Moreover, titles or subtitles may be used in the specification for the convenience of a reader, which has no influence on the scope of the invention. Additionally, some terms used in this specification are more specifically defined below.

### DEFINITIONS

The terms used in this specification generally have their ordinary meanings in the art, within the context of the invention, and in the specific context where each term is used.

Certain terms that are used to describe the invention are discussed below, or elsewhere in the specification, to provide additional guidance to the practitioner in describing the apparatus and methods of the invention and how to make and use them. For convenience, certain terms may be highlighted, for example using italics and/or quotation marks. The use of highlighting has no influence on the scope and meaning of a term; the scope and meaning of a term is the same, in the same context, whether or not it is highlighted. It will be appreciated that the same thing can be said in more than one way. Consequently, alternative language and synonyms may be used for any one or more of the terms discussed herein, nor is any special significance to be placed upon whether or not a term is elaborated or discussed herein. Synonyms for certain terms are provided. A recital of one or more synonyms does not exclude the use of other synonyms. The use of examples anywhere in this specification, including examples of any terms discussed herein, is illustrative only, and in no way limits the scope and meaning of the invention or of any exemplified term. Likewise, the invention is not limited to various embodiments given in this specification. Furthermore, subtitles may be used to help a reader of the specification to read through the specification, which the usage of subtitles, however, has no influence on the scope of the invention.

As used herein, “around”, “about” or “approximately” shall generally mean within 20 percent, preferably within 10 percent, and more preferably within 5 percent of a given value or range. Numerical quantities given herein are approximate, meaning that the term “around”, “about” or “approximately” can be inferred if not expressly stated.

As used herein, the term “scanning electron microscope (SEM)” refers to a type of electron microscope that images the sample surface by scanning it with a high-energy beam of electrons in a raster scan pattern. The electrons interact with the atoms that make up the sample producing signals that contain information about the sample's surface topography, composition and other properties such as electrical conductivity.

As used herein, the term “X-ray diffraction (XRD)” refers to one of X-ray scattering techniques that are a family of non-destructive analytical techniques which reveal information about the crystallographic structure, chemical composition, and physical properties of materials and thin films. These techniques are based on observing the scattered intensity of an X-ray beam hitting a sample as a function of incident and scattered angle, polarization, and wavelength or energy. In particular, X-ray diffraction finds the geometry or shape of a molecule, compound, or material using X-rays. X-ray diffraction techniques are based on the elastic scattering of X-rays from structures that have long range order.

As used herein, the term “Transmission electron microscopy (TEM)” refers to a microscopy technique whereby a beam of electrons is transmitted through an ultra thin specimen, interacting with the specimen as it passes through. An image is formed from the interaction of the electrons transmitted through the specimen; the image is magnified and focused onto an imaging device, such as a fluorescent screen, on a layer of photographic film, or to be detected by a sensor such as a CCD camera. In particular, TEMs are capable of imaging at a significantly higher resolution than light microscopes, owing to the small de Broglie wavelength of electrons. This enables the instrument to be able to examine fine detail—even as small as a single column of atoms, which is tens of thousands times smaller than the smallest resolvable object in a light microscope.

As used herein, the term “Magnetic Resonance Imaging (MRI)” refers to a medical imaging technique most commonly used in radiology to visualize the internal structure and function of the body. MRI provides much greater contrast between the different soft tissues of the body than computed tomography (CT) does, making it especially useful in neurological (brain), musculoskeletal, cardiovascular, and oncological (cancer) imaging. Unlike CT, it uses no ionizing radiation, but uses a powerful magnetic field to align the nuclear magnetization of (usually) hydrogen atoms in water in the body. Radio frequency (RF) fields are used to systematically alter the alignment of this magnetization, causing the hydrogen nuclei to produce a rotating magnetic field detectable by the scanner. This signal can be manipulated by additional magnetic fields to build up enough information to construct an image of the body.

As used herein, “nanoscopic-scale,” “nanoscopic,” “nanometer-scale,” “nanoscale,” the “nano-” prefix, and the like generally refers to elements or articles or stuctures having widths or diameters of less than about 1 μm, preferably less than about 100 nm in some cases. In all embodiments, specified widths can be smallest width (i.e. a width as specified where, at that location, the article can have a larger width in a different dimension), or largest width (i.e. where, at that location, the article's width is no wider than as specified, but can have a length that is greater).

As used herein, “plurality” means two or more.

As used herein, the terms “comprising,” “including,” “carrying,” “having,” “containing,” “involving,” and the like are to be understood to be open-ended, i.e., to mean including but not limited to.

### OVERVIEW OF THE INVENTION

While the applications of nanoparticles have been continuously expanded, major efforts have also been devoted to provide these nanoparticles with sufficient protection against such degradations, by encasing them into inert chemical components. For example, carbon, inorganic compounds or surfactant and polymers are among the most commonly used materials for coating such nanoparticles used for biomedical applications. These coatings besides their protective roles, also offer means of attaching the complex structures to biological systems such as antibodies, proteins, DNA, etc in order to target particular cell lines like cancer.

The present invention, in one aspect, relates to metallic nanoparticles coated with graphitic shells, and a novel method or process for using them as localized radio frequency (“RF”) absorbers for cancer therapy.

In some embodiments, to show the enablement and utility of the invention, although many cells or cell lines can be chosen, HeLa cells were used since they proliferate abnormally fast when compared to normal or other cancer cells and represent great models for studying the interactions between nanosystems and cancerous cells.

In some embodiments, to show the enablement and utility of the invention, graphitic carbon-coated ferromagnetic cobalt nanoparticles (“C—Co-NPs”) with diameters of around 7 nm and cubic crystalline structures were synthesized by catalytic chemical vapor deposition. The C—Co-NPs may also be synthesized by other methods or processes known to people skilled in the art. As formed, X-ray diffraction and x-ray photoelectron spectroscopy analysis indicated that the cobalt nanoparticles inside the carbon shells were preserved in the metallic state. Fluorescence microscopy images and Raman spectroscopy revealed effective penetrations of the C—Co-NPs through the cellular plasma membrane of the cultured HeLa cells, both inside the cytoplasm and in the nucleus. Low radio frequency (RF) radiation of 350 kHz induced localized heat from the metallic nanoparticles, which triggered the killing of the cells, a process that was found to be dependent on the RF application time and nanoparticle concentration. When compared to carbon nanostructures such as single-wall carbon nanotubes, these coated magnetic cobalt nanoparticles demonstrated higher specificity for RF absorption and heating. DNA gel electrophoresis assays of the HeLa cells after the RF treatment showed a strong broadening of the DNA fragmentation spectrum, which further proved the intense localized thermally induced damages such as DNA and nucleus membrane disintegration, under RF exposure in the presence of C—Co-NPs. The data presented in this specification thus indicate current utility and a great potential of this invention for in vivo tumor thermal ablation, bacteria killing, and various other biomedical applications.

Moreover, RF resonance heating is less invasive and possesses higher efficiency for targeting localized cells or sub-cellular compartments, and thus is effective to reduce the side effects associated with the traditional cancer therapies. Previous experiments showed that thermal ablation by means of electromagnetic radiation energy could reliably create foci of tissue necrosis as large as 1.6 cm in diameter. However, most tumors are significantly larger and their possible detection time delays, successful treatments have, until recently, necessitated the use of either multiple treatment probes, or multiple treatment sessions, or a combination of both. Therefore, a major focus of research has been on the development of techniques for achieving single-session large-volume tissue necrosis in a safe and readily accomplished manner [13]. In some embodiments, The C—Co-NPs synthesized by a standard catalytic chemical vapor deposition (CCVD) method have been found to act as RF absorbers and tissue temperature inducers, mechanism which can be developed into a more sensitive and reliable tumor targeting and successful thermal ablation process. The process was used for targeting the cancerous cells, intracellular delivery of the C—Co-NPs and the inducement of apoptosis under RF excitation. This process can be extended to in vivo tumor targeting if these nanoparticles are attached to antibodies, proteins, or other such delivery vehicles. Also the delivery of magnetic nanoparticles to relatively large tumor regions can be done directly by self-delivery or by injection while the localized heating driven by RF could be responsible for the tumor ablation process. The thermal results induced by the C—Co-NPs under exposure to low frequency RF radiation have been compared to the results obtained in identical conditions but when single-wall carbon nanotubes were used as the thermal agents. The cell work has been extended to understanding the mechanism that is responsible for the death of the cells by identifying the localized thermal damages such as DNA fragmentation associated with this process. Such medical therapies also can be applied to bacterial, viruses or other biological systems and hold promise for successful tumor treatments in medical clinical applications.

Accordingly, the present invention, in one aspect, relates to a novel method or process for treatment of cancer.

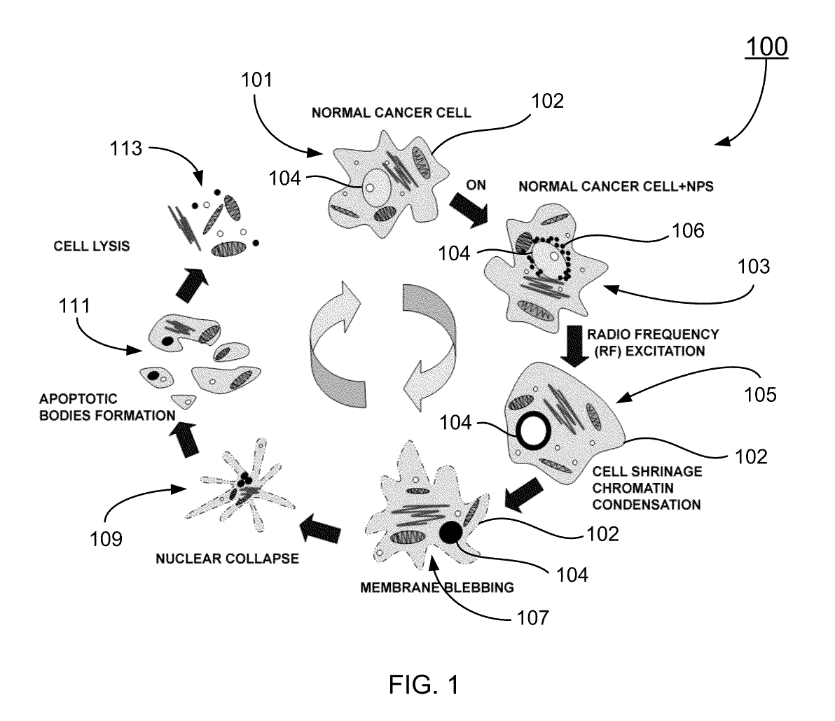

More specifically, referring now to FIG. 1, a method 100, according to one embodiment of the present invention, includes one or more steps as follows: at step 101, tissure 102 with a cancer cell 104 is identified. Then at step 103, a plurality of metallic nanoparticles 106 is provided, wherein each of the plurality of metallic nanoparticles has a core formed with a first metallic material, and a coated shell formed with a non-metallic material containing carbon, and wherein the coated shell is formed to enclose the metallic core completely; and introduced into the cancel cell 104 such that said metallic nanoparticles 106 selectively target cancer cell 104. Subsequently at step 105, at least one radio frequency of electromagnetic waves is applied to tissue 102 for a period of time effective to induce skin currents in the cores of the first metallic material of said metallic nanoparticles 106 to cause heat generated locally around targeted cancer cell 104. Under the heat locally generated by the said metallic nanoparticles 106, the membrane of cancer cell 104 starts blebbing at step (time line) 107, the nuclear of cancer cell 104 collapses at step (time line) 109, cancer cell 104 shows apoptotic bodies formation at step (time line) 111, and eventually, cancer cell 104 shows cell lysis or is dead at step (time line) 113.

These and other aspects of the present invention are further described below.

### EXAMPLES AND IMPLEMENTATIONS OF THE INVENTION

Without intent to limit the scope of the invention, exemplary methods and their related results according to the embodiments of the present invention are given below. Note again that titles or subtitles may be used in the examples for convenience of a reader, which in no way should limit the scope of the invention. Moreover, certain theories are proposed and disclosed herein; however, in no way they, whether they are right or wrong, should limit the scope of the invention.

### Example 1

This example describes how C—Co-NPs were made according to one embodiment of the present invention.

C—Co-NPs were prepared by utilizing a urea combustion method. Mg(NO3)2·6H2O, Co(NO3)2·6H2 and urea (all supplied by Aldrich, purity >99%) were mixed together in the stoichiometric amounts. The mixture slurry was heated in a crucible at 100° C. to thoroughly dehydrate. Such dried mixture is capable of igniting easily while placed into a crucible. Typically, the combustion reaction was completed after only 15 min and the powdered materials was grinded and stored in an oven at 110° C. to dry completely. About 3 g of the Co—MgO solid was set in the frit of a quartz tube centered inside a vertical tubular furnace. A H2/CH4 mixture (18 mol % of CH4) was introduced over the catalyst at 600° C. for 10 min and the reaction was continued for 30 min at 800° C. The C—Co—MgO product was finally obtained by exposure to concentrated HCl to dissolve the MgO support and the non-carbon-coated Co nanoparticles. The C—Co-NPs were separated by washing with deionized water through a 0.5 μm polycarbonate membrane using a Millipore filtration rig and then dried in air at 110° C.

Transmission electron microscopy (TEM) images of the C—Co-NPs were collected on an H-9500 TEM (Hitachi High-Technologies Corp) with acceleration voltage of 300 kV. In this example, C—Co-NPs powder was dispersed in 2-propanol and ultrasonicated for 10 min. A few drops of the suspension were deposited on a TEM grid, then dried, and evacuated before analysis. X-ray diffraction (XRD) and x-ray photoelectron spectroscopy (XPS) X-ray diffraction of Co nanoparticles coated with carbon shells were recorded on a Bruker AXS D8 advanced diffractometer (Cu Kα) in θ/2θ geometry with a general area detector. The patterns were recorded over 10°<2θ<80°. The phase identifications were performed with EVA software. XPS measurements were performed using a Thermo Scientific KAlpha at a background pressure of 1×10⁻⁹ Torr, using a monochromated Al Kα (hv=1456.6 eV) x-ray source and a combined low-energy electron/ion flood gun for charge neutralization. The collected data were referenced to the graphite C1s peak to 284.5 eV [15]. Detection limits for XPS are approximately 0.1-1.0 at. % depending upon the sensitivity of the elements. Raman scattering spectra of the catalysts and CNTs were collected at room temperature on Horiba Jobin Yvon LabRam HR800 equipped with a charge-coupled detector and a spectrometer with a 600 lines mm⁻¹ grating. He—Ne (633 nm, 2.41 eV) laser was used as the excitation source. The laser beam intensity measured at the sample was kept at 5 mW. Raman shifts were calibrated with a silicon wafer at the peak of 521 cm⁻¹.

### Example 2

This example describes sample cells or cell culture used in some embodiment of the present invention. The present invention can be practiced with respect to other type of cells or cell cultures as well.

For the cell culture, mammalian cervical cancer cells (HeLa cells) were seeded in 10 cm2 culture plates (0.5×106 cells/plate) with growth medium (minimum essential medium containing 10% fetal bovine serum and 1% penicillin 100 unit ml⁻¹, streptomycin 100 μg ml⁻¹) and incubated in a humidified incubator (37° C., 5% CO2). For the subculture, cells were dissociated by 1× trypsin/EDTA in PBS and counted and plated into 35 mm culture plates at a density of 5×104 cells/plate and supplemented by growth medium with various concentrations of C—Co-NPs (0.83-20 μg ml⁻¹) and without C—Co-NPs for control (0 μg ml⁻¹ Vehicle).

### Example 3

This example describes Sample preparations for the C—Co-NPs and single-wall carbon nanotubes with cells or cell cultures, respectively, according to some embodiments of the present invention, and certain related measurements.

The C—Co-NPs and single-wall carbon nanotubes were administered, respectively, to the cells in identical concentrations as the C—Co-NPs in order to compare the effects of the two species of nanostructures.

For acridine orange/ethidium bromide staining, cells were washed with PBS (10 mM, pH 7.4) and stained with a solution of, 100 mg ml⁻¹ acridine orange and 100 mg ml⁻¹ ethidium bromide in PBS and mixed together in a ratio of 1:1. Cells were then visualized immediately under UV light using Olympus fluorescence microscope at 10× objective equipped with a digital camera. Photographs were taken using randomly selected fields of view. To determine the percentage of cells undergoing apoptosis, photographs taken were used for counting the number of live (green) and apoptotic (orange) cells. Acridine orange stains live cells green whereas ethidium bromide stains fragmented nuclear DNA in dead cells as red. Approximately 200-300 cells per treatment were counted to obtain statistical rate of cell death. Cells were subjected to 350 kHz, 5 kW of radio frequency induction for time periods ranging from 2 to 45 min. The proliferation of the cells was done under fluorescence microscopy counting the dead and alive cells. C—Co-NPs powder and powder dispersed in medium solution were set in the

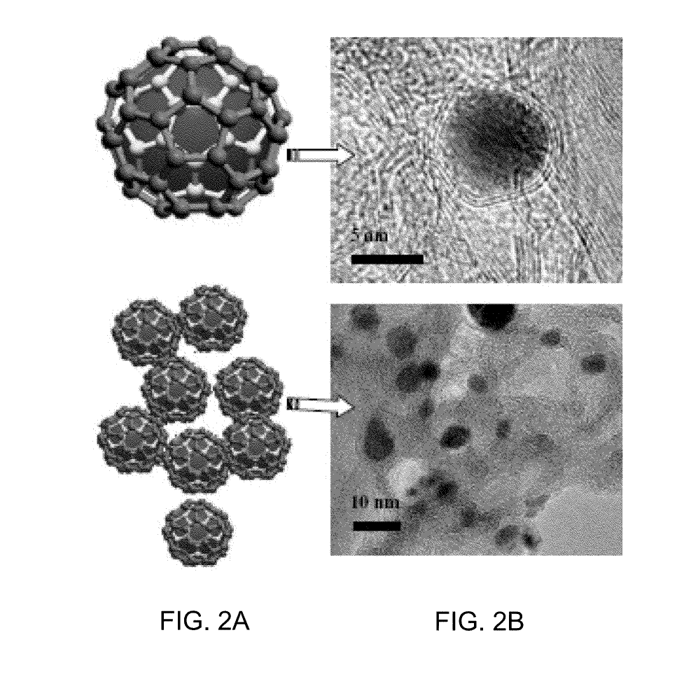

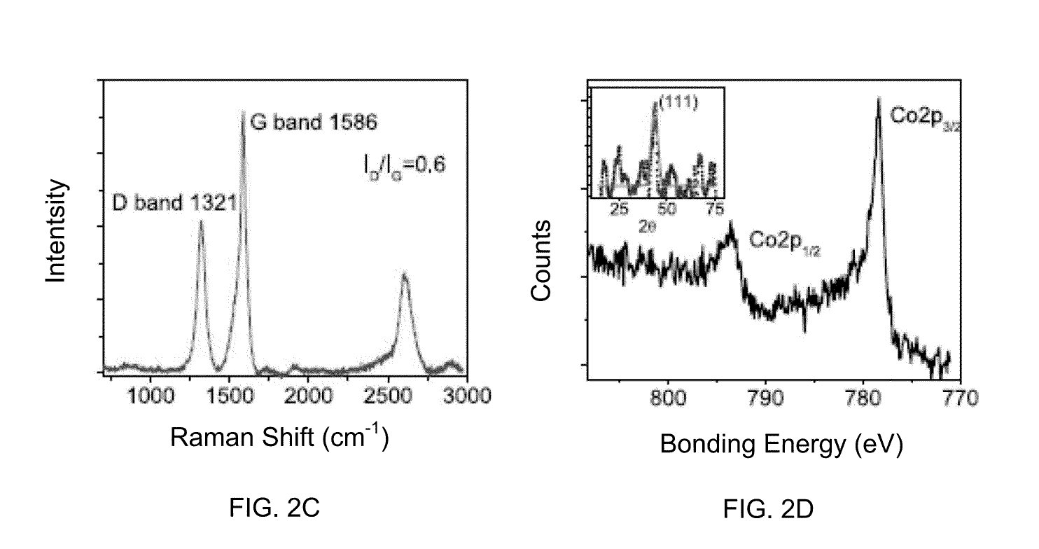

Petri dish individually and induced by RF heating for 5 min. Before and after RF heating, a Thermometer (PTM 01, Russia) was used to check the temperature of powder surface or the solution. C—Co-NPs were synthesized by a regular CCVD process. TGA analysis indicates the presence of 20% of cobalt NP encapsulated by crystalline graphitic shells mixed with singlewall carbon nanotubes. The separation of these two species was carried out as described previously. TEM analysis of over 200 images revealed that the average size of the nanocrystals was 7±1.2 nm and they were covered with 2-4 layers of graphitic carbon atoms, as shown in FIGS. 2(a) and (b) [15, 18]. The oxides of Co, particularly CoO and Co3O4, show significant shake-up satellite peaks at about 5-6 eV in binding energy above the main Co 2p3/2 peak, which are absent in the spectrum presented in FIG. 2(d). Also, the Co-oxides are noticeably upshifted in binding energy to about 780-781 eV, compared to metallic Co at 778.35.

The O 1s spectrum of Co-oxides consists of two peaks: 529-531 eV. The O 1s peak was measured at 532.35 eV, considerably higher than that for Co-oxides and may be attributed to O—C, or O═C bonding [19], which means that the cobalt is kept in the metallic state in these nanostructures.

The XRD profile in shows that the face-centered-cubic fcc structure phase is the predominant phase of the Co-NPs. Since the crystalline sizes are so small, only the strongest fcc lattice (111) cobalt peak domains at around 2θ=44. are visible in the XRD spectra. The existence of the cobalt peak corresponding to the non-oxidized metallic state suggests that Co-NPs are uniformly covered by the graphitic layers, which prevented the metal from reacting with HCl during the purification stage. No peaks corresponding to the oxidized state of the nanostructures were found by XRD. The nanoparticles translocation in vitro into cells most probably happened due to already studied processes but also by diffusion, trans-membrane channels, or adhesive interactions [20]. Among the surface charges between cells and nanoparticles, particle types, and sizes; the size was found to be the most important factor for the cells translocation. Here, the C—Co-NPs<10 nm were visualized to aggregate around the membrane of nucleus and then penetrate the nuclear membrane to nucleus after being dispersed individually in the phosphate-buffered saline (PBS) medium solution used to feed the HeLa cells for 24 h.

### Example 4

This example describes an exemplary process according to one embodiment of the present invention.

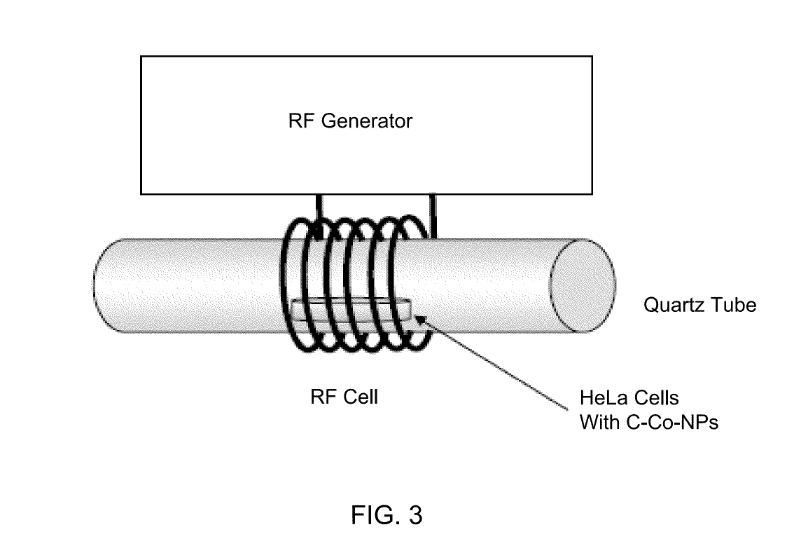

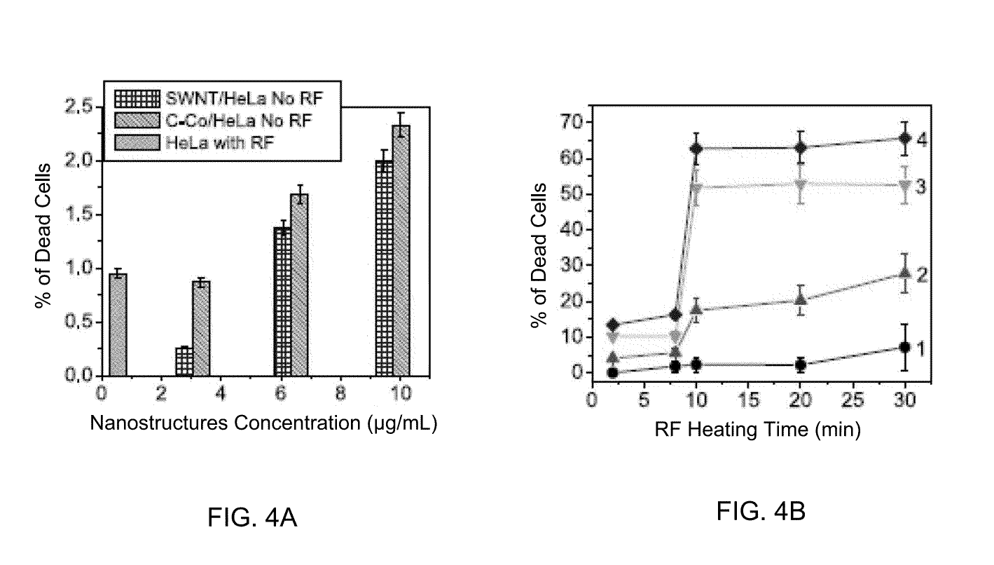

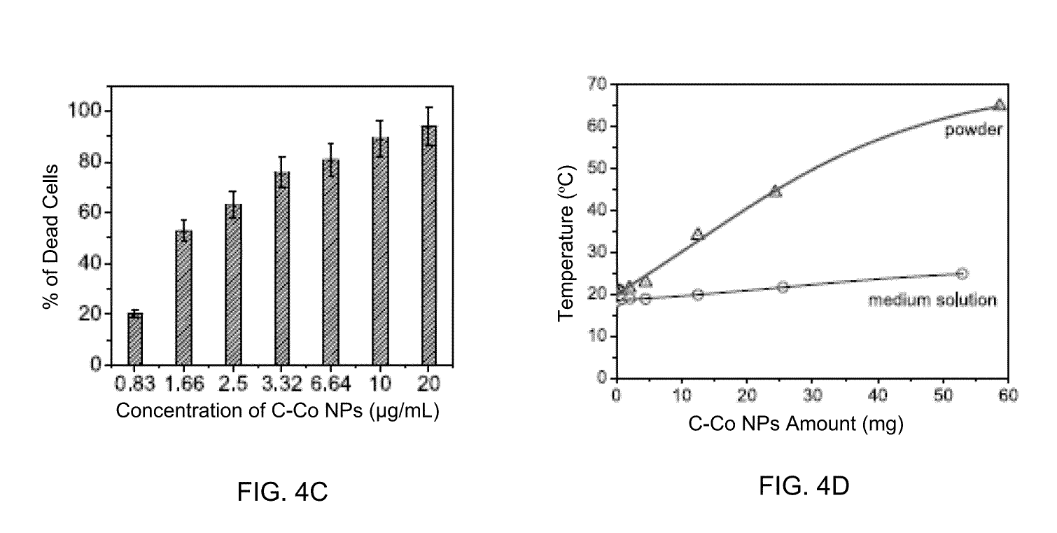

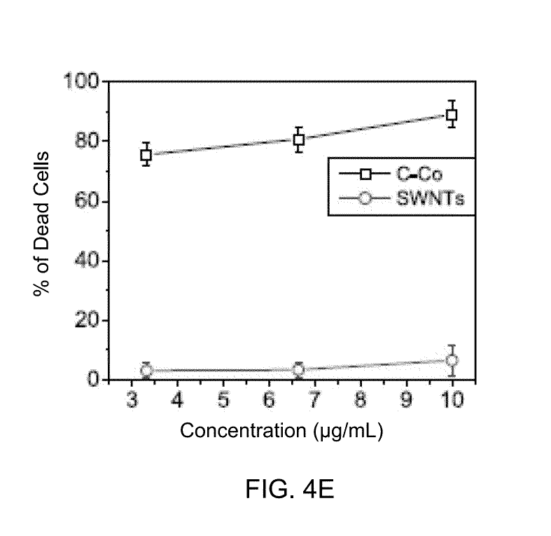

Cells cultured with the nanoparticles, which were prepared as set forth in one or more of Examples 1-3 set forth above, at different concentrations and various time periods, were introduced inside a water-cooled coil coupled to a radio frequency generator (Pillar, Tex.) with the frequency of 350 kHz (as showed in the schematic FIG. 3(a)), which is far lower than what was the commonly used range of between 10 MHz and 300 GHz [22]. Such low frequency radiation has the ability to penetrate the biological tissues efficiently and present a path of cancer treatment deep inside the body (such as at 400 kHz, field penetration into 15 cm of tissue is >99%) [23]. For this exemplary embodiment, the frequency was chosen as 350 kHz. The present invention, however, can be practiced at least in a range of 10 to 500 kHz. Cytotoxicity studies have indicated that only 0.9% to 2.3% of the total cultured cells incubated with different concentrations ranging from 3.3 to 10 μμg ml⁻¹ of C—Co-NPs, were found dead, which indicated a low level of toxicity of C—Co-NPs (in FIG. 4(a)). However, the toxicity of the nanomaterials inside the living systems is still a disputed topic today. The data presented in FIG. 4(a) also shows that the RF alone induced hardly any effect to the HeLa cells with only around 1% of the cells dead.

After the RF heating, the total numbers of dead and alive cells were immediately counted following fluorescence staining and visualization by fluorescence microscopy. FIG. 4(b) shows the statistical results of the influence of the RF exposure on the inducement of apoptosis in the cells, treated for time periods ranging from 2 to 30 min. After 8 min of RF exposure, the death rate of the cells increased drastically. This finding indicated the existence of a critical time point (exposure time) or exposure time threshold at which the cells die at a high rate. For a concentration of 2.5 μg ml⁻¹ C—Co-NPs delivered into the HeLa cells medium solution, around 63% of the cells were found to die after 10 min of RF heating, while only around 16% cells died within 8 min of RF heating and 13% died after 2 min of RF exposure. Approximately 10 min of exposures were required to substantially increase the number of dead cells for a given concentration of nanoparticles. Moreover, for RF exposure times longer than 10 min, the percentage of dead cells increased rather slow and in some cases almost remained relatively constant. Therefore, about 10 min of RF exposure seems to be the time that has the maximum effect on the cell-killing rate for the particular cell sample here.

As expected, the concentration of C—Co-NPs also plays a very important role in the inducement of apoptosis as shown in FIG. 3, where RF excitation setup (350 kHz, 5 kW) was used for the thermal ablation of HeLa cells. RF energy, given the super paramagnetic properties of cobalt, as shown in FIG. 4(c), increasing the cobalt nanoparticles concentration was reflected in a higher numbers of cells killed. For such a concentration of 20 μg ml ⁻¹, up to 98% of the cancer cells (in total 105 cells) became apoptotic and self-degraded after a few minutes of RF treatment. The results obtained can be explained by the fact that the RF electromagnetic radiation induces skin currents (heat) in the C—Co-NPs to allow them to generate heat and thus increases their temperature due to Ohmic effects.

### Example 5

This example describes certain studies related to the heating effect in connection with Example 4 according to some embodiments of the present invention.

In order to investigate the heating effect inside the HeLa cells, the temperature changes in nanopowders form and nanopowders dispersed in solution under RF were studied. Comparatively the surface temperature rise for the C—Co-NPs powder and in the medium solution with different concentrations under RF heating for 5 min was continuously monitored and was shown in FIG. 4(d). Five minutes were considered sufficient in order to highlight the difference in the heating characteristics of the C—Co-NPs when in powder or individually dispersed in a solution. The high sensitivity thermal analysis indicated that the RF induced temperatures (up to 70° C.) into the C—Co-NPs powders as well as their heating rates were found to be dependent upon mostly the mass of the nanomaterials used for the measurements.

On the contrary, when the nanoparticles were individually suspended in the medium solution, no major bulk heating was observed and the temperature remained almost constant (only from room temperature to 25° C.) with increasing the concentration of the C—Co-NPs.

### Example 6

This section provides various aspects of some exemplary embodiments of the present invention set forth in EXAMPLES 1-5.

From the experimental results set forth above, since the mass difference between the nanoparticles present inside the cells and the cells themselves is significant, the death of the cells is not expected to happen due to the bulk heating of the entire cell structures, but rather due to the localized damage of the membranes (especially of the nucleus), DNA fragmentation, and protein thermal damages and denaturation (that happen at temperatures higher than 55° C.). These studies are in good correlation with previous studies that have reported that for in vivo heating of up to 42° C., there would be required about 1.2 mg particles in a 1 cm³ tissue volume [26]. In the previous study of [26], however, the Co-NPs concentration was too low (around 3.3-20 μg ml⁻¹) to allow exact temperature measurements of the nanoparticles taken up inside the cells.

The heat transfer from the nanoparticles to the solution is mostly governed by the heat transfer equations, and since the dimension of the nanoparticles (about 10 nm for single nanoparticle, or several hundreds nanometer to micrometer size when they aggregated together) compared to that of the solution is extremely small, the overall solution temperature was rarely increased. However, the RF radiation was found to be absorbed nanoparticles and they were heated up and created the localzed damages in various cellular sub-compartments (10-50 μm), which induced the death of the cells. Besides the already presented thermally induced effects of the RF irradiation into the magnetic nanoparticles, such type of electromagnetic radiation was also reported to be responsible for making the tissues in general and the cells in particular to be more susceptible to radiation or chemotherapy, due to the localized heating and breakage of the nuclear membranes that allows a more readily administration of drug molecules.

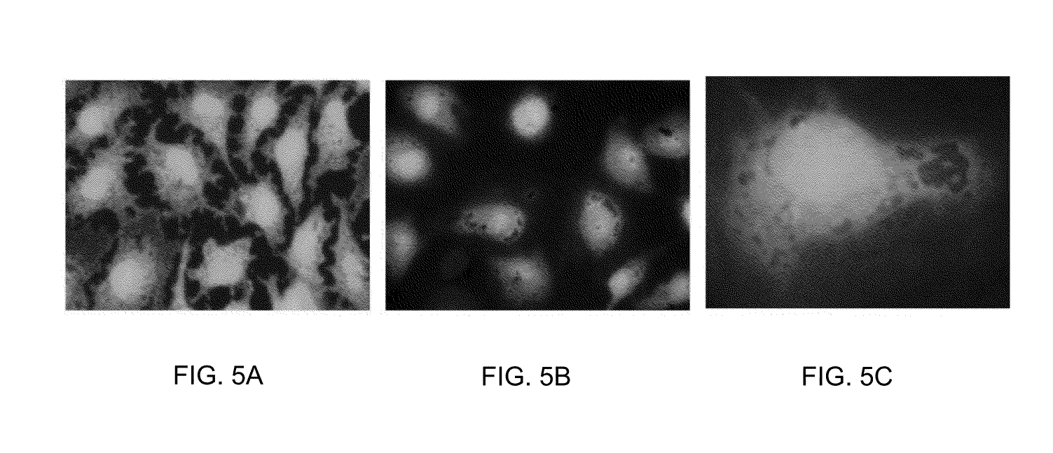

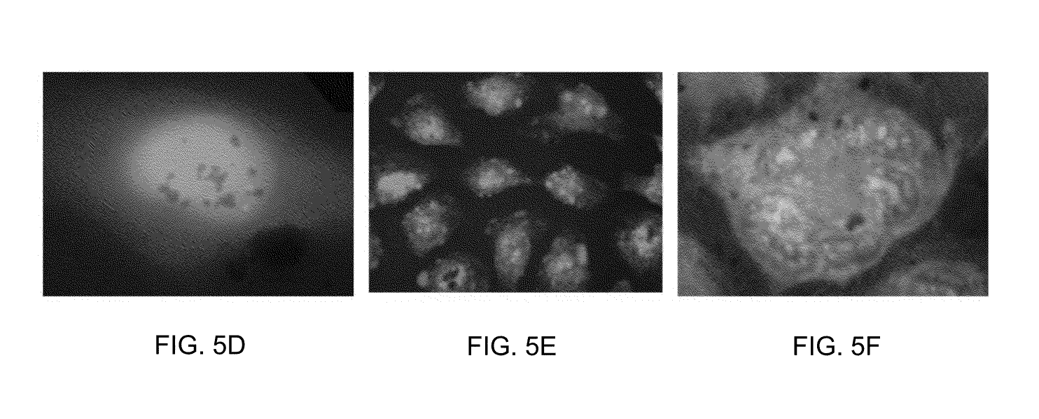

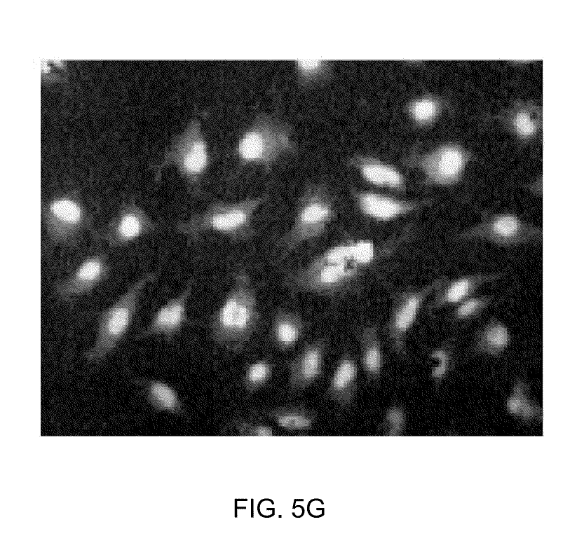

After nanoparticles were uptaken into the cells cytoplasm, they were found to agglomerate around the nuclear membrane (as shown in FIG. 5(c)), and a small number crossed the nuclear membrane into the nucleus (as shown in FIG. 5(d)). Raman spectroscopy results further confirmed that nanoparticles were inside the nucleus and in larger quantities inside the cytoplasm next to the nuclear membrane. Therefore, these nanoparticles have the ability to cross the various inter-cellular membranes and to reach the nucleus (as shown in FIG. 2). Due to the localized RF heating provided by the nanoparticles, the cells were found to go through an accelerated apoptotic process and consequently cellular decomposition, as shown in FIGS. 5(e)-(g).

To further identify the function of the ferromagnetic metal nanoparticles, single-wall carbon nanotubes (SWNTs) were used as a comparison. SWNTs were synthesized on a bimetallic catalyst system Fe—Mo supported on MgO and were grown by RF-CCVD method using acetylene as the carbon source [28]. The dominant diameter distribution ranges from 0.7 to 2 nm (as showed in FIG. 3). Exactly identical concentrations of

SWNTs (from 3.3 to 10 μg ml⁻¹) were incubated with HeLa cells in similar conditions as the Co-NPs. Cellular cytotoxicity studies of SWNTs showed a low toxicity as indicated in FIG. 4(a). After the cells were exposed to RF radiation for 20 min in identical conditions as done in the Co-NPs case, only 3.1-6.6% of the cells were found to be dead versus 75.2-90.1% with Co-NPs (FIG. 4(e)). This increase rate is significantly lower, demonstrating the more intense RF absorption and heat generation by the metallic Co nanoparticles compared to the SWNTs. This result is further highlighted by the real-time IR tomography heating analysis (as shown in FIG. 4).

It was accordingly found that the more enhanced heating of the metallic nanoparticles under identical RF radiation compared to the SWNTs, because the temperature rose much higher than SWNTs. The disintegration of nano-localized cell environments such as nucleus, nuclear membranes, and DNA is believed to be the main effect of the RF heat inducement into the Co nanoparticles.

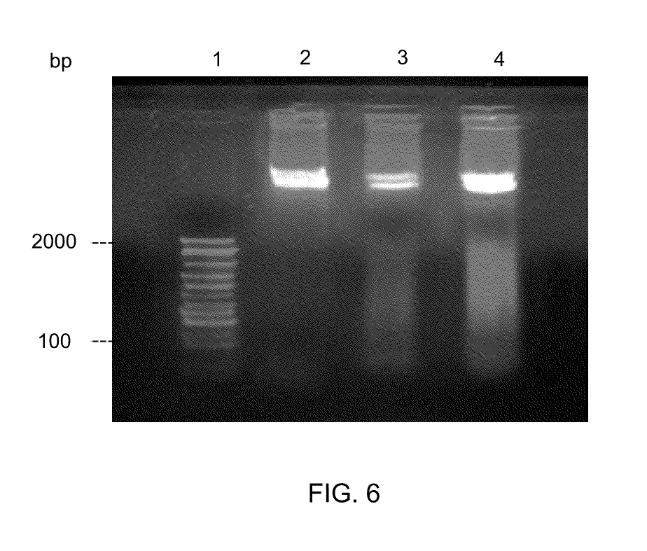

FIGS. 5(e) and (f) show the disintegration of the nucleus membranes in the RF heating process. This initial apoptosis screening process was then followed by additional analysis, as cellular morphology studies using agarose gel electrophoresis to detect oligonucleosomal ladders, which is an effect of the apoptosis inducement into cells. From the gel electrophoresis analysis (as shown in FIG. 6), it can be observed that comparatively to the cells kept as control, the DNA collected from the cells exposed to the cobalt nanoparticles and RF exposure showed the strongest broadening of the DNA spectrum. The nuclear DNA was degraded into fragments of about 2000-100 by which was shown in FIG. 6. These results indicate chemical modifications [29] of the DNA bases and the breakage of the DNA double strands process that can induce mitotic recombinations, point mutations, and chromosomes loss and translocation. Therefore, these results indicate that such magnetic nanoparticles can become strong RF absorbers and therefore can generate thermally localized cellular damages such as DNA fragmentation and breakage of the cellular membranes, which can induce cell death and cancerous tissue necrosis.

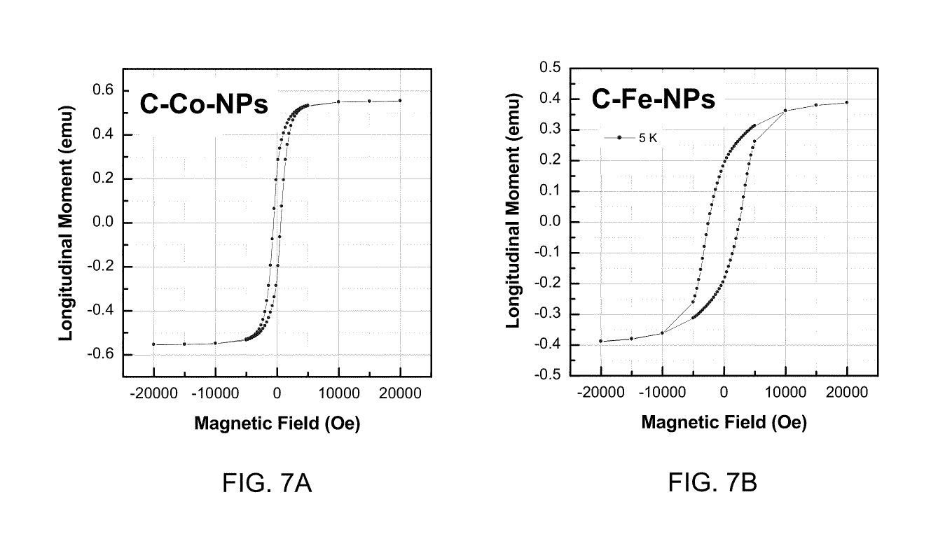

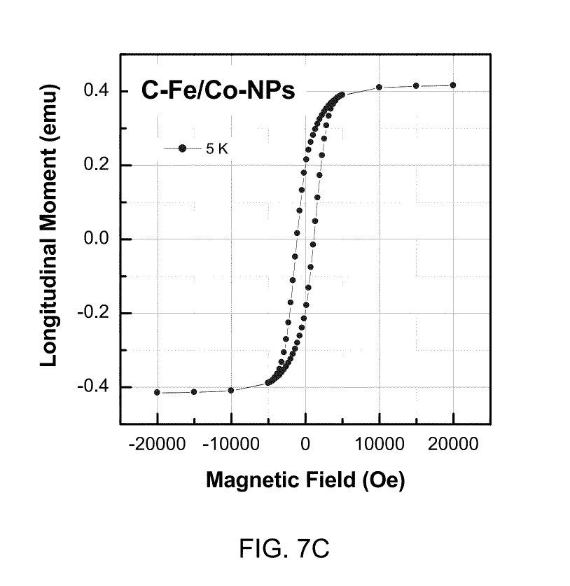

The magnetic properties of the C—Co-NPs, the C—Fe-NPs and the C—Fe/Co-NPs are shown in FIG. 7, respectively. Note that, like Co, Fe (or other metallic materials) can be utilized to form the metallic (magnetic) core of the invented nanoparticle.

The delivery of high enough concentrations of such magnetic nanoparticles can be done by means of antibodies, proteins, or other targeting biological systems to the tumor sites and their thermal excitation under exposure to RF, xrays or other types of electromagnetic radiations. The low RF frequency used according to the present invention could be essential for the thermal ablation treatment of deep cancer tumors, which so far has proved difficult to achieve.

In sum, the present invention presents the successful use of hybrid advanced nanomaterials with magnetic cores and covered by several graphitic shells. These materials have significant advantages over the regular magnetic nanoparticles, since the metallic core is never exposed to the liquid biological environments and therefore their structural, magnetic, optic, and spectroscopic properties are not expected to change over time but stay in a metallic state. Moreover, this lack of contact between the Co nanostructures and the biological systems is expected to limit their potential toxic effect due to reduced metal leaking into blood or tissues. The graphitic shells which have strong Raman signal (as shown in FIG. 2(c)) could allow the detection of such nanostructures and their real-time in vivo biodistribution analysis by Raman flow cytometry or by MRI. By the intermediation of surface group functionalities that can be attached to the graphitic top layers, these nanoparticles have the potential to be attached to cancer specific antibodies or proteins for direct delivery to tumors or even individual cells in circulation in blood or lymph. Specific delivery of such nanomaterials into tumors or to individual cells allows them to be killed by applying low frequency RF radiation, which has a relatively large penetration inside the tissues. In vivo and in vitro experiments are currently being carried out to further such an approach for cancer thermal ablation.

The foregoing description of the exemplary embodiments of the invention has been presented only for the purposes of illustration and description and is not intended to be exhaustive or to limit the invention to the precise forms disclosed. Many modifications and variations are possible in light of the above teaching.

The embodiments were chosen and described in order to explain the principles of the invention and their practical application so as to activate others skilled in the art to utilize the invention and various embodiments and with various modifications as are suited to the particular use contemplated. Alternative embodiments will become apparent to those skilled in the art to which the present invention pertains without departing from its spirit and scope. For example, multiple probes may be utilized at the same time to practice the present invention. Accordingly, the scope of the present invention is defined by the appended claims rather than the foregoing description and the exemplary embodiments described therein.

### LIST OF REFERENCES

[1] Paciotti G F, Myer L, Weinreich D, Goia D, Pavel N, McLaughlin R E and Tamarkin L 2004 Colloidal gold: a novel nanoparticle vector for tumor directed drug delivery Drug Deliv. 11 169-83.
[2] Jain K K 2005 Nanotechnology-based drug delivery for cancer Technol. Cancer Res. Treat. 4 407-16.
[3] Alivisatos P 2004 The use of nanocrystals in biological detection Nat. Biotechnol. 22 47-52.
[4] Wu X, Liu H, Liu J, Haley K N, Treadway J A, Larson J P, Ge N, Peale F and Bruchez M P 2003 Immunofluorescent labeling of cancer marker Her2 and other cellular targets with semiconductor quantum dots Nat. Biotechnol. 21 41-6.
[5] Everts M et al 2006 Covalently linked Au nanoparticles to a viral vector: potential for combined photothermal and gene cancer therapy Nano Lett. 6 587-91.
[6] Zharov V P, Galitovskaya E N, Johnson C and Kelly T 2005 Synergistic enhancement of selective nanophotothermolysis with gold nanoclusters: potential for cancer therapy Lasers Surg. Med. 37 219-26.
[7] Hirsch L R, Stafford R J, Bankson J A, Sershen S R, Rivera B, Rrice R E, Hazle J D, Halas N J and West J L 2003 Nanoshell-mediated near infrared thermal thermal therapy of 8 Nanotechnology 19 (2008) 435102 Y Xu et al tumors under magnetic resonance guidance Proc. Natl Acad Sci. USA 100 13549-54.
[8] Seo W et al 2006 FeCo/graphitic-shell nanocrystals as advanced magnetic-resonance-imaging and near-infrared agents Nat. Mater. 5 971-6.
[9] Mornet S, Vasseur S, Grasset F and Duguet E 2004 Magnetic nanoparticle design for medical diagnosis and therapy J. Mater. Chem. 14 2161-75.
[10] Sun S, Murray C B, Weller D, Folks L and Moser A 2000 Monodisperse FePt nanoparticles and ferromagnetic FePt nanocrystal superlattices Science 287 1989-92.
[11] H\ddot{}utten A, Sudfeld D, Ennen I, Reiss G, HachmannbW, Heinzmann U, Wojczykowski K, Jutzi P, Saikaly W and Thomas G 2004 New magnetic nanoparticles for biotechnology J. Biotechnol. 112 47-63.
[12] Rutnakornpituk M, Thompson M S, Harris L A, Farmer K E, Esker A R, Riffle J S, Connolly J and St Pierre T G 2002 Formation of cobalt nanoparticle dispersions in the presence of polysiloxane block copolymers Pine Bluff. 43 2337-48
[13 ] Stevenson J P, Rutnakornpituk M, Vadala M, Esker A R, Riffle J S, Charles S W, Wells S and Dailey J P 2001 Magnetic cobalt dispersions in poly(dimethylsiloxane) fluids J. Magn. Magn. Mater. 225 47-58.
[14] Scott G G, Nahum G S, Luigi S and Tito L 2000 Tumor ablation with radio-frequency energy Radiology 217 633-46.
[15] Flahaut E, Peigney A, Laurent Ch and Rousset A 2000 Synthesis of single-walled carbon nanotube-Co—MgO composite powders and extraction of the nanotubes J. Mater. Chem. 10 249-52.
[16] Moulder J F, Stickle W F, Sobol P E and Bomben K D 1992 Handbook of X-ray Photoelectron Spectroscopy ed J Chastain (Eden Prairie, Minn.: Perkin-Elmer Corporation).
[17] Sato K, Saito R, Oyama Y, Jiang J, Cancado L G, Pimenta M A, Jorio A, Samsonidze Ge G, Dresselhaus G and Dresselhaus M S 2006 D-band Raman intensity of graphitic materials as a function of laser energy and crystallite size Chem. Phys. Lett. 427 117-21.
[18] Flahaut E, Agnoli F, Sloan J, O'Connor C and Green M L H 2002 CCVD synthesis and characterization of cobalt-encapsulated nanoparticles Chem. Mater. 14 2553-8.
[19] McIntyre N S and Cook M G 1975 X-ray photoelectron studies on some oxides and hydroxides of cobalt, nickel, and copper Anal. Chem. 47 2208-13.
[20] Briggs D and Seah M P 1999 Practical Surface Analysis by Auger and X-ray Photoelectron Spectroscopy 2nd edn (Chichester: Wiley).

[21] Geiser M, Rothen-Rutishauser B, Kapp N, Schurch S, Kreyling W, Schulz H, Semmler M, ImHof V, Heyder J and Gehr P 2005 Ultrafine particles cross cellular membranes by nonphagocytic mechanisms in lungs and in cultured cells Environ. Health Perspect. 113 1555-60.
[22] Rothen-Rutishauser B M, Schurch S, Haenni B, Kapp N and Gehr P 2006 Interaction of fine particles and nanoparticles with red blood cells visualized with advanced microscopic techniques Environ. Sci. Technol. 40 4353-9.
[23] Leea S et al 2005 2.45 GHz radiofrequency fields alter gene expression in cultured human cells FEBS Lett. 579 4829-36.
[24] Young J H, Wang M T and Brezovich I A 1980 Frequency/depth-penetration considerations in hyperthermia by magnetically induced currents Electron Lett. 16 358-9.
[25] Biris A S et al 2007 Influence of the RF excitation of the catalyst system on the morphology of multiwalled carbon nanotubes J. Phys. Chem. C 111 17970-5.
[26] Little R B, Biris A R, Lupu D, Xu Y, Li Z, Dervishi E and Biris A S 2008 On the dynamical ferromagnetic, quantum Hall, and relativistic effects on the carbon nanotubes nucleation and growth mechanism J. Magn. Magn. Mater. 320 540-7.
[27] Derfus A M, Maltzahn G, Harris T J, Duza T, Vecchio K S, Ruoslahti E and Bhatia S N 2007 Remotely triggered release from magnetic nanoparticles Adv. Mater. 19 3932-6.
[28] Xiang X, Wang G, Yang H, Melman J and Dionne G F 2007 Remotely RF powered conformable thermal applicators US Patent Specification 0169001.
[29] Biris A R, Biris A S, Dervishi E, Lupu D, Trigwell S, Rahman Z and Marginean P 2006 Catalyst excitation by radio frequency for improved carbon nanotubes synthesis Chem. Phys. Lett. 429 204-8.
[30] Zhu L, Chang D W, Dai L and Hong Y 2007 DNA damage induced by multiwalled carbon nanotubes in mouse embryonic stem cells Nano Lett. 7 3592-7.

What is claimed is:

1. A nanostructure, comprising:
(a) a core formed with a first metallic material, wherein the core has a diameter in the range of 5 to 10 nm; and
(b) a shell formed with a second material that is different from the first metallic material,
wherein the shell is formed to enclose the metallic core and has a thickness of at least two layers of atoms of said second material.

2. The nanostructure of claim 1, wherein said core is adapted to absorb at least one radio frequency of electromagnetic waves when said core is subject to the radiation of said electromagnetic waves.

3. The nanostructure of claim 1 usable as a localized RF absorber for cancer therapy.

4. The nanostructure of claim 1, wherein the first metallic material is selected from the group consisting of Co, Sb, Li, Rb, Ti, V, Mn, Fe, Ni, Cu, Zn, Zr, Mo, Ru, Rh, Pd, Ag, W, Ir, Pt, and a combination thereof.

5. The nanostructure of claim 1, wherein the second material is selected from the group consisting of non-metal materials containing carbon, noble metallic materials, and polymeric materials.

6. The nanostructure of claim 1 usable as a MRI contrast agent.

7. The nanostructure of claim 1 usable as a delivery vehicle for drug and biological systems that include growth factors, antibodies, genes, DNA, RNA and a combination of them to a targeted area.

8. The nanostructure of claim 1, wherein said core is adapted to absorb laser radiation or electromagnetic radiation when said core is subject to the laser radiation or electromagnetic radiation.
